(19) United States
(12) Patent Application Publication
(10) Pub. No.: US 2023/0151194 A1
(43) Pub. Date: May 18, 2023

(54) ANAEROBIC BIODEGRADATION ACCELERATOR FOR POLYMERIC MATERIALS, METHODS FOR PRODUCING AND USING THEREOF

(71) Applicant: Nano and Advanced Materials Institute Limited, Hong Kong (HK)

(72) Inventors: Yeuk Tin LAU, Hong Kong (HK); Lisi HE, Hong Kong (HK); Sui Han SIN, Hong Kong (HK); Guozhuangqi YAN, Hong Kong (HK); Kwan Wing CHEU, Hong Kong (HK); Shing Hang NG, Hong Kong (HK); Kin Hang Ken NG, Hong Kong (HK)

(21) Appl. No.: 17/987,879

(22) Filed: Nov. 16, 2022

Related U.S. Application Data
(60) Provisional application No. 63/280,625, filed on Nov. 18, 2021.

(51) Int. Cl. C08L 23/06; C12N 11/08

(52) U.S. Cl. CPC C08L 23/06; C12N 11/08; C08L 2207/066

(57) ABSTRACT

An anaerobic biodegradation accelerator (ABA) for a host polymeric material, an ABA-incorporated polymeric material, and methods for production and application thereof are provided. The ABA includes a carrier matrix, at least one biotic component, a protective layer, a biodiversity promotor, a surfactant, a compatibilizer, an antioxidant, a plasticizer and a properties modifier. The ABA significantly enhances biodegradation rate of polymeric materials in anaerobic environments, and does not impact significantly on mechanical properties and other properties of the original polymeric material including food contact safety when they are used in food contact safe products such as cutleries, lunch boxes, cups and cup lids.

### CROSS-REFERENCE TO RELATED APPLICATIONS

The present application claims priority from U.S. provisional patent application Ser. No. 63/280,625 filed Nov. 18, 2021, and the disclosure of which is incorporated herein by reference in its entirety.

### FIELD OF THE INVENTION

The present invention relates to a biodegradation accelerator for accelerating biodegradation of host polymeric materials in the presence of biotic materials under various conditions, in particular, under anaerobic conditions. The present invention also relates to methods for producing an anaerobic biodegradation accelerator and using thereof.

### BACKGROUND

Reducing the use of plastic is one way to reduce plastic waste, but it appears that one of the most compromised ways to reduce the same nowadays is by an effective plastic degradation. To achieve this, one of the most popular methods in plastic degradation is by adding an additive called oxo-degradable additive into the polymeric materials. However, oxo-degradable additives are going to be banned by more and more counties or regions, such as France, Spain and New Zealand, because their biodegradability are in doubt and the microfragmentation/microplastic resulting therefrom can be even more harmful to our environment. Therefore, some recent studies focused on exploring possible alternatives for enhancing the biodegradability of conventional plastics.

US patent application publication number US2013/0109781 A1 disclosed a chemical additive, which uses furanone compounds as chemoattractants, and the additive material is claimed to be blended with polymeric material to create at least a partially biodegradable product. However, detail of how these chemoattractants interact with microbes to facilitate biodegradation in extreme environment, e.g., in low or substantially oxygen-free and high temperature environment, was not provided, nor how to shorten the biodegradation time or improve the efficiency thereof by modifying the common plastics. Also, quorum sensing is claimed to be used to attract bacteria to the polymer in order to enhance the biodegradability of the polymer. This would not change the population dynamics of the treated site. Instead, additional microbes will be included in the chemical additive for the purpose of attempting to change the population dynamics. However, the additional microbes may not be compatible with the indigenous microbes and may be faded out in a short period of time.

In addition, food-contact safe aspect and other practical use of the end products are also not fully considered in the prior arts. Therefore, there is a need for a formulation/method to accelerate biodegradation of polymeric materials, considering the long-term biodegradation, biodiversity promotor will be instead included in the formulation to modify the indigenous microbial population so as to facilitate the biodegradation, including conventional plastic and common biodegradable plastics.

### SUMMARY OF THE INVENTION:

Accordingly, in a first aspect, the present invention provides an anaerobic biodegradation accelerator (ABA) for a host polymeric material, and the ABA includes:

a carrier matrix for gathering all other ingredients in an accelerator and assisting in dispersing them into the host polymeric material,

at least one biotic component for initiating the biodegradation of the host polymeric material,

a protective layer for protecting the biotic component and increasing shelf-life of the ABA,

a biodiversity promotor for promoting and sustaining growth of the at least one biotic component,

a surfactant for promoting the interaction of the at least one biotic component and the host polymeric material,

a compatibilizer to increase the compatibility between the accelerator and the host polymeric material,

an antioxidant for inhibiting oxidation reaction of the accelerator during manufacturing, storage, and usage,

a plasticizer, and

a properties modifier.

In an embodiment, the carrier matrix includes, but not limited to, biodegradable and/or non-biodegradable materials selected from one or more of polyethylene (PE), polypropylene (PP), poly (ethylene-vinyl acetate) (EVA), polystyrene (PS), polyoxymethylene (POM), polyethylene terephthalate (PET), polyethylene terephthalate glycol (PETG), polyamides (PA), polycarbonate (PC), polyurethanes (PU), thermoplastic elastomer (TPE), cellulose acetate (CA), polyvinyl chloride (PVC), acrylonitrile butadiene styrene (ABS), polyvinyl alcohol (PVA), polylactic acid (PLA), polyhydroxyalkanoates (PHAs), polybutylene succinate (PBS), polycaprolactone (PCL), polybutylene adipate terephthalate (PBAT), polyglycolic acid (PGA), and poly(lactic-co-glycolic acid) (PLGA), or any combination thereof.

The carrier matrix can be in an amount of 30% to 90% of the total weight of the anaerobic biodegradation accelerator.

In an embodiment, the at least one biotic component is selected from bacteria, fungi, and enzymes, or any combination thereof.

The bacteria include, but not limited to, Clostridium thermocellum, Micrococcus luteus, Rhodococcus rhodochrous, Streptomyces badius, Acinetobacter spp., Alcaligenes spp., Amycolatopsis spp., Arthrobacter spp., Bacillus spp., Citrobacter spp. Corynebacterium spp., Enterobacter spp., Exiguobacterium spp., Lysinibacillus spp., Bacillus megaterium, Bacillus subtilis, Microbacterium spp., Micrococcus spp., Nocardia spp., Paenibacillus spp., Pseudomonas spp., Rhodococcus spp., Schlegelella spp., Sphingobacterium spp., and Staphylococcus spp.

The fungi include, but not limited to, yeast, Aspergillus niger, Acremonium spp., Aspergillus spp., Aureobasidium spp., Cladosporium spp., Fusarium spp., Gliocladium spp., Mucor spp., Penicillium spp., Pestalotiopsis spp., Phanerochaete spp., Streptomyces spp. Trametes spp., and Trichoderma spp.

The enzymes include, but not limited to, α-amylase, catalase, cellulase, cutinase, depolymerase, esterase, glucosidases, hydrolase, laccase, lipase, manganese peroxidase, urease, protease such as papain, bromelain.

The at least one biotic component can be in an amount of greater than 0% to 20% of the total weight of the anaerobic biodegradation accelerator.

In an embodiment, the protective layer includes one or more protective layer materials of gum arabic, sodium alginate, gelatin, chitosan, cellulose, polyvinyl alcohol, poly(lactic-co-glycolic acid), polyethylene glycol, or any combination thereof, and further incorporates the surfactant.

The one or more protective layer materials is/are in an amount of greater than 0% to 30% of the total weight of the anaerobic biodegradation accelerator.

In an embodiment, the biodiversity promotor includes, but not limited to, saccharide compounds, nitrogen-containing compounds, phosphorous compounds, or any derivatives thereof, and micronutrients, and wherein the saccharide compounds include cyclodextrins, cellulose, starch, sucrose, and glucose; the nitrogen-containing compounds include proteins, meat extracts, autolysates, nitrates, and urea; the phosphorous compounds include phosphorus pentoxide, hydrogen phosphates, dihydrogen phosphate, and organic phosphate; the derivatives include pectin, xylan, carboxylic acids, amino acids; and the micronutrients include vitamins, minerals, potassium, calcium, magnesium, iron, manganese, zinc, boron, copper, and molybdenum; or any combination thereof

The biodiversity promotor can be in an amount of greater than 0% to 20% of the total weight of the anaerobic biodegradation accelerator.

In various embodiment, the surfactant is one or more of non-ionic or ionic surfactants, and wherein the non-ionic surfactants include polysorbates, sorbitan esters, and alkylphenol ethoxylates; the ionic surfactants includes cationic surfactants, anionic surfactants, zwitterionic surfactants, and biosurfactants, and wherein the anionic surfactants include anionic functional group-containing compounds includes sulfate, sulfonate, phosphate, carboxylate derivatives, prominent alkyl sulfates include ammonium lauryl sulfate, sodium lauryl sulfate and the related alkyl-ether sulfates sodium laureth sulfate and sodium myreth sulfate, dioctyl sodium sulfosuccinate, perfluorooctanesulfonate, perfluorobutanesulfonate, alkyl-aryl ether phosphates, alkyl ether phosphates sodium dodecylbenzenesulfonate, sodium dodecyl sulfate, sodium stearate, calcium stearate; the cationic surfactants include octenidine dihydrochloride, cetrimonium bromide, cetylpyridinium chloride, benzalkonium chloride, benzethonium chloride, dimethyldioctadecyl ammonium chloride, and dioctadecyldimethylammonium; the zwitterionic surfactants include lauryldimethylamine oxide and myristamine oxide; the biosurfactants include glycolipids, phospholipids, lipopeptides, neutral lipids, fatty acids, and lipopolysaccharides; or any combination thereof.

The surfactant in different embodiments can be in an amount of greater than 0% to 10% of the total weight of the anaerobic biodegradation accelerator.

In an embodiment, the compatibilizer includes, but not limited to, chain extenders and coupling agents, and wherein the chain extenders include modified styrene acrylic polymers, lactic acid, ethylene glycol, and 1,4-butanediol; the coupling agents include maleic anhydride, Tung oil anhydride, epoxidized soybean oil, methylene-diphenyldiisocyanate, acrylic acid, and citric acid; or any combination thereof

The compatibilizer can be in an amount of greater than 0% to 10% of the total weight of the anaerobic biodegradation accelerator.

In an embodiment, the plasticizer includes, but not limited to, water, urea, glycerol, ethylene glycol, polyethylene glycol, Tung oil anhydride, epoxidized soybean oil, triethyl citrate, and acetyl triethyl citrate, or any combination thereof

The plasticizer can be in an amount of greater than 0% to 10% of the total weight of the anaerobic biodegradation accelerator.

In an embodiment, the properties modifier includes, but not limited to, calcium carbonate, titanium dioxide, talcum powder, organomontmorillonite, bentonite, nanofillers, natural fiber, color masterbatch, and scent masterbatch, or any combination thereof.

The properties modifier can be in an amount of greater than 0% to 10% of the total weight of the anaerobic biodegradation accelerator.

In an embodiment, the antioxidant includes, but not limited to, ascorbic acid, tocopherols, glutathione, tetrakis [methylene(3,5-di-t-butyl-4-hydroxyhydrocinnamate)]methane, tris(2,4-di-tert-butylphenyl) phosphite, lipoic acid, and uric acid, or any combination thereof.

The antioxidant can be in an amount of greater than 0% to 10% of the total weight of the anaerobic biodegradation accelerator.

In an embodiment, the host polymeric material includes, but not limited to, polyethylene (PE), polypropylene (PP), poly (ethylene-vinyl acetate) (EVA), polystyrene (PS), polyoxymethylene (POM), polyethylene terephthalate (PET), polyethylene terephthalate glycol (PETG), polyamides (PA), polycarbonate (PC), polyurethanes (PU), thermoplastic elastomer (TPE), cellulose acetate (CA), polyvinyl chloride (PVC), acrylonitrile butadiene styrene (ABS), polyvinyl alcohol (PVA), polylactic acid (PLA), polyhydroxyalkanoates (PHAs), polybutylene succinate (PBS), polycaprolactone (PCL), polybutylene adipate terephthalate (PBAT), polyglycolic acid (PGA), and poly(lactic-co-glycolic acid) (PLGA), or any combination thereof, and wherein the host polymeric material is conventional plastic or any processed plastic by common plastic technologies, including, but not limited to, extrusion, resin making, foaming, sheet production, thermoforming, injection molding, film blowing, blow molding, fiber/fabric and filament making.

In a second aspect, there is provided a method for producing the anaerobic biodegradation accelerator in the first aspect as a masterbatch, and the method includes:

introducing the protective layer material to the at least one biotic component to form a protective layer surrounding the at least one biotic component;

homogenizing the at least one biotic component with protective layer and the remaining components and/or materials of the anaerobic biodegradation accelerator at a first elevated temperature to obtain a mixture; and

extruding the mixture under a second elevated temperature until the masterbatch is obtained.

The first elevated temperature can range from room temperature to about 80° C.

The second elevated temperature can range from 50° C. to about 250° C.

The homogenization of the at least one biotic component with protective layer and the remaining components and/or materials can be carried out at a mixing speed of 40 to 1,000 rpm.

In a third aspect, there is provided a method for producing an anaerobic biodegradation accelerator (ABA)-incorporated polymeric material incorporated with the anaerobic biodegradation accelerator of foregoing aspects in a masterbatch form, where the method includes:

homogenizing the masterbatch of the anaerobic biodegradation accelerator (ABA) with a host polymeric material to form a blend; and

extruding the blend at a third elevated temperature to obtain an anaerobic biodegradation accelerator-incorporated polymeric material.

In an embodiment, the anaerobic biodegradation accelerator in the ABA-incorporated polymeric material is in an amount of greater than 0% to 30% by weight.

More preferably, the anaerobic biodegradation accelerator is in an amount of about 1% to 5% by weight of the ABA-incorporated polymeric material in order to achieve a minimal affection of mechanical properties, and other properties of the original polymeric material including food contact safety when they are used in food contact safe products.

The third elevated temperature can range from 50° C. to about 300° C.

The homogenization of the masterbatch of the ABA with the host polymeric material can be carried out at a mixing speed of 40 to 1,000 rpm.

The ABA-incorporated polymeric material of the present invention has a recyclability comparable to that of the host polymeric material.

The ABA-incorporated polymer material also has a superior biodegradability than that of the host polymeric material.

The ABA-incorporated polymeric material includes, but not limited to, polyethylene (PE), polyoxymethylene (POM), polystyrene (PS), expanded polystyrene (EPS), polypropylene (PP) and polyethylene terephthalate (PET) which is incorporated with the anaerobic biodegradation accelerator of the present invention.

### BRIEF DESCRIPTION OF THE DRAWINGS:

Embodiments of the invention are described in more details hereinafter with reference to the drawings, in which:

FIG. 1 schematically depicts the conceptual illustration of an anaerobic biodegradation accelerator (ABA) according to an embodiment of the present invention;

FIG. 2 schematically depicts how ABA accelerates biodegradation of polymeric material in anaerobic environment according to an embodiment of the present invention;

FIG. 3 schematically depicts a method of producing an ABA masterbatch according to an embodiment of the present invention;

FIG. 4 schematically depicts a method of producing an ABA-incorporated polymeric material according to an embodiment of the present invention, and illustrates some of their applications;

FIGS. 5A-5C depict an ABA-incorporated low-density polyethylene (LDPE) produced according to an embodiment of the present invention; FIG. 5A depicts a photo showing a film of ABA-incorporated LDPE by extrusion-film blowing process; FIG. 5B demonstrates a photo showing an appearance of a roll of film of LDPE (left) and that of ABA-incorporated LDPE produced according to FIG. 5A incorporating 5 wt. % ABA (right); FIG. 5C shows a photo of a plastic bag produced by the 5 wt. % ABA-incorporated LDPE according to the roll of film shown in FIG. 5B;

FIG. 6 shows appearance of different products made of different host polymers incorporated with 5 wt. % ABA produced according to an embodiment of the present invention;

FIG. 7 shows the food contact safety test report of an ABA-incorporated fork;

FIG. 8 shows the difference in biodegradation rate of LDPE with or without incorporation of 5 wt. % ABA during a 480-day period; and

FIG. 9 shows the difference in biodegradation rate of POM with or without incorporation of 5 wt. % ABA during a 450-day period.

### DETAILED DESCRIPTION OF THE INVENTION:

In the following description, the formulations, compositions and methods for producing and using the same, and the likes, are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

As used herein, the term “greater than 0%” refers to any percentage that is higher than 0%, including 0.0001%, 0.001%, 0.01%, 0.1% and 1%.

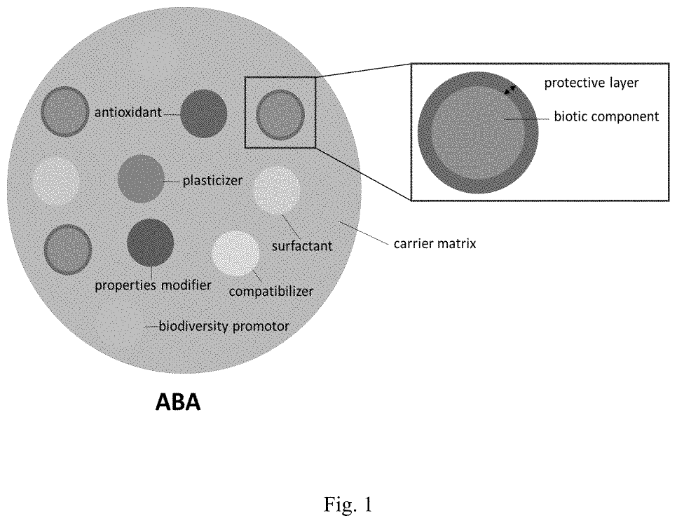

Turning to the drawings, FIG. 1 depicts the conceptual illustration of key components of the present anaerobic biodegradation accelerator (ABA), including a carrier matrix, a biodiversity promotor included in the carrier matrix, biotic component protected by a protective layer, surfactant, compatibilizer, plasticizer, antioxidant, and properties modifier.

The carrier matrix in the present invention is used for gathering all other ingredients in the ABA and assisting in dispersing them into the polymeric materials in which the ABA is incorporated, where the carrier matrix the carrier matrix includes, but not limited to, biodegradable and/or non-biodegradable materials selected from one or more of polyethylene (PE), polypropylene (PP), poly (ethylene-vinyl acetate) (EVA), polystyrene (PS), polyoxymethylene (POM), polyethylene terephthalate (PET), polyethylene terephthalate glycol (PETG), polyamides (PA), polycarbonate (PC), polyurethanes (PU), thermoplastic elastomer (TPE), cellulose acetate (CA), polyvinyl chloride (PVC), acrylonitrile butadiene styrene (ABS), polyvinyl alcohol (PVA), polylactic acid (PLA), polyhydroxyalkanoates (PHAs), polybutylene succinate (PBS), polycaprolactone (PCL), polybutylene adipate terephthalate (PBAT), polyglycolic acid (PGA), and poly(lactic-co-glycolic acid) (PLGA), or any combination thereof. In some embodiments, the carrier matrix in the ABA is present in the amount from approximately 30% to less than 100% by weight of the ABA, and preferably from approximately 30% to 90% by weight of the ABA. In a preferred embodiment, the carrier matrix is composed of biodegradable materials to enhance the initial biodegradability and growth of microbes for further biodegradation.

In certain embodiments, the biotic component can be bacteria selected from strictly anaerobe or facultative anaerobe, for example, Clostridium thermocellum, Micrococcus luteus, Rhodococcus rhodochrous, Streptomyces badius, Acinetobacter spp., Alcaligenes spp., Amycolatopsis spp., Arthrobacter spp., Bacillus spp., Citrobacter spp. Corynebacterium spp., Enterobacter spp., Exiguobacterium spp., Lysinibacillus spp., Bacillus megaterium, Bacillus subtilis, Microbacterium spp., Micrococcus spp., Nocardia spp., Paenibacillus spp., Pseudomonas spp., Rhodococcus spp., Schlegelella spp., Sphingobacterium spp., Staphylococcus spp., etc.; fungi, for example, Yeast, Aspergillus niger, Acremonium spp., Aspergillus spp., Aureobasidium spp., Cladosporium spp., Fusarium spp., Glioclodium spp., Mucor spp., Penicillium spp., Pestalotiopsis spp., Phanerochaete spp., Streptomyces spp. Trametes spp., Trichoderma spp., etc. The biotic component can also be enzymes, for example, α-amylase, catalase, cellulase, cutinase, depolymerase, esterase, glucosidases, hydrolase, laccase, lipase, manganese peroxidase, protease (for example papain, bromelain), urease, etc. In some embodiment, the biotic component in the ABA is present in the amount from greater than 0% to less than 70% by weight of the ABA, and preferably from greater than 0% to 20% by weight of the ABA.

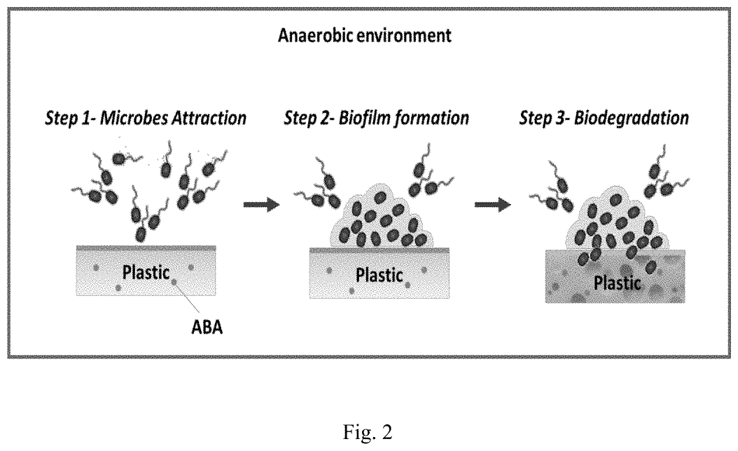

In certain embodiments, the surfactant is used for promoting the interaction of microorganisms (or the biotic component) with the plastics (e.g., hydrophobic plastics) to accelerate the microbial attachment to polymeric materials and the biofilm formation, such as the mechanism as shown in FIG. 2, where microbes are attracted by the plastic incorporated with the ABA of the present invention (step 1) and forms a biofilm more efficiently than the original plastic without ABA (step 2), followed by accelerating the biodegradation of the plastic (step 3). The surfactant of the present invention can include, but not limited to, the following materials: nonionic surfactants, such as polysorbates, sorbitan esters, alkylphenol ethoxylates; ionic surfactants, anionic surfactants (for example, surfactants containing anionic functional groups at their head, sulfate, sulfonate, and phosphate, carboxylate derivatives, prominent alkyl sulfates include ammonium lauryl sulfate, sodium lauryl sulfate and the related alkyl-ether sulfates sodium laureth sulfate and sodium myreth sulfate, dioctyl sodium sulfosuccinate, perfluorooctanesulfonate, perfluorobutanesulfonate, alkyl-aryl ether phosphates, alkyl ether phosphates sodium dodecylbenzenesulfonate, sodium dodecyl sulfate, sodium stearate, calcium stearate, etc.); cationic surfactants (for example, octenidine dihydrochloride, cetrimonium bromide, cetylpyridinium chloride, benzalkonium chloride, benzethonium chloride, dim ethyl di octadecyl amm onium chloride, and di octadecyl dim ethyl amm onium bromide etc.); Zwitterionic surfactants (for example lauryl dim ethyl amine oxide and myristamine oxide, etc.); bi o surfactants (for example, glycolipids, phospholipids, lipopeptides, neutral lipids, fatty acids, and lipopolysaccharides, etc.). In some embodiments, the surfactant in the ABA is present in the amount from greater than 0% to 10% by weight of the ABA.

In certain embodiments, the compatibilizer is used for increasing the compatibility between the immiscible components in the ABA, and also increasing the compatibility between the accelerator and the polymeric materials to achieve a high dispersion of ABA in the polymeric materials. The compatibilizer of the present invention can include, but not limited to, the following materials: chain extenders, such as modified styrene acrylic polymers, lactic acid, ethylene glycol, 1,4-butanediol; coupling agents, such as maleic anhydride, Tung oil anhydride, epoxidized soybean oil, methylene-diphenyldiisocyanate, acrylic acid, citric acid, etc. In some embodiment, the compatibilizers in the ABA is present in the amount from greater than 0% to 10% by weight of the ABA.

In certain embodiments, the antioxidant is used for inhibiting oxidation reaction to protect the ABA during the thermo-processing such as extrusion, and to increase the shelf life the ABA during storage and usage. The antioxidant of the present invention can include, but not limited to, the following materials: ascorbic acid, tocopherol s, glutathione, tetrakis [methylene(3 ,5-di-t-butyl hydroxyhydrocinnamate)]methane, tris(2,4-di-tert-butylphenyl) phosphite, lipoic acid, uric acid, etc. In some embodiments, the antioxidant in the ABA is present in the amount from greater than 0% to 10% by weight of the ABA.

In certain embodiments, the plasticizer of the present invention is used for increasing the plasticity of the accelerators to facilitate the manufacture process. The plasticizer includes, but not limited to, the following materials: water, urea, glycerol, ethylene glycol, polyethylene glycol, Tung oil anhydride, epoxidized soybean oil, triethyl citrate, acetyl triethyl citrate, etc. In some embodiment, the plasticizers in the ABA is present in the amount from greater than 0% to 10% by weight of the ABA.

In certain embodiments, the properties modifier of the present invention is used for improving specific properties, such as mechanical properties, of the ABA and/or introducing specific properties, such as color and odor, to the polymeric materials, in which the anaerobic biodegradability is to be enhanced. In those embodiments, the properties modifier can include, but not limited to, the following materials: calcium carbonate, titanium dioxide, talcum powder, organomontmorillonite, bentonite, nanofillers, natural fiber, color masterbatch, scent masterbatch, etc. In some embodiments, the properties modifier in the ABA is present in the amount from greater than 0% to 10% by weight of the ABA.

In certain embodiments, the protective layer of the present invention is used for protecting the biotic component during the production of the ABA and for minimizing migration thereof when the ABA is incorporated into food contact safe products such as cutleries, while the shelf-life of the ABA in terms of storage and usage is increased. In some embodiments, the protective layer is made of one or more materials, preferably be biodegradable, to enhance the initial biodegradability and growth of microbes for further biodegradation. The protective layer material of the present invention can include, but not limited to, the following materials: gum arabic, sodium alginate, gelatin, chitosan, cellulose, polyvinyl alcohol, poly(lactic-co-glycolic acid), polyethylene glycol. The protective layer further incorporates one or more surfactants. In some embodiments, the protective layer material in the ABA is present in the amount from greater than 0% to 30% by weight of the ABA, and preferably from approximately greater than 0% to 10% by weight of the ABA.

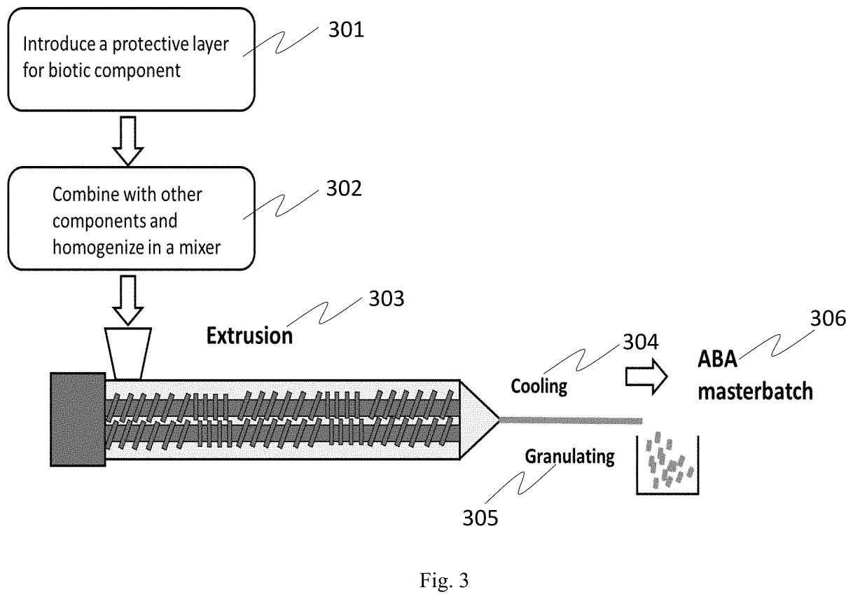

Further, the present ABA is prepared in a masterbatch form by a method shown in FIG. 3. As in FIG. 3, protective layer material and biotic component are initially introduced into a homogenizer (301) by various techniques such as spray drying, extrusion method, emulsion, spray chilling, coacervation, cocrystallization, liposome formation, etc., followed by adding other components of the ABA into the homogenizer to mix thoroughly (302), before being subjected to an extruder for extrusion (303). The mixing speed of the homogenizer ranges from about 40 to 1,000 rpm, and the operation temperature of the homogenizer ranges from room temperature to about 80° C. when the components of ABA are mixed in the homogenizer. The thoroughly mixed ABA components are then extruded in the extruder at an operation temperature ranges from 50° C. to about 250° C. After extrusion, the extruded ABA will be subjected to cooling (304), and then granulation (305), to form the ABA masterbatch (306). It should be understood that any reasonable modifications, variations and optimization of the production method, or any other available methods known to a skilled artisan, can also be used to produce the present ABA masterbatch, provided that the resulting ABA masterbatch meets the desired biodegradability in the polymeric materials after incorporation thereof into the polymeric materials.

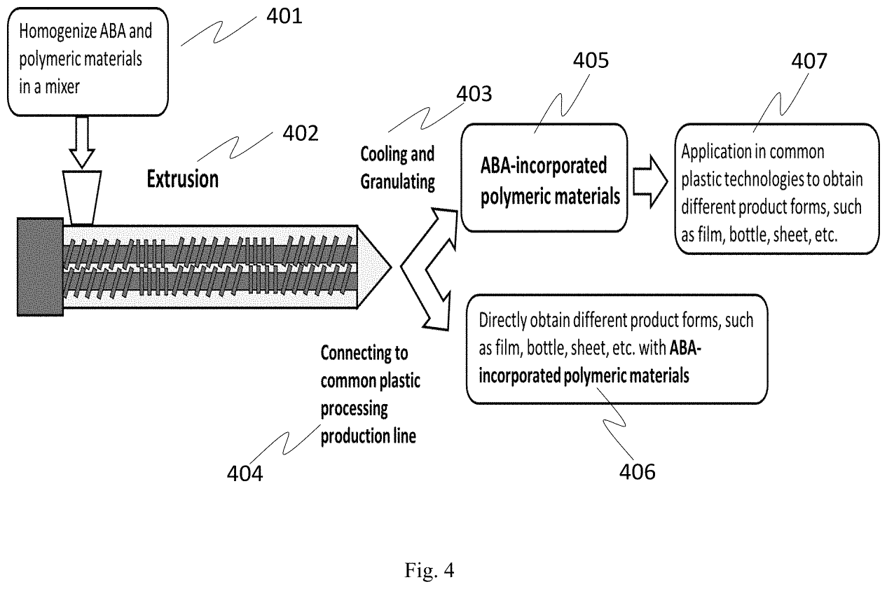

FIG. 4 illustrates how the ABA according to certain embodiments of the present invention, such as the ABA masterbatch produced according to the method as shown in FIG. 3, is incorporated into polymeric materials to produce an ABA-incorporated polymeric material, and what possible products/applications can the ABA-incorporated polymeric materials be made into or used for, such as resins, films, bottles, cups, boxes, cutleries, foams, etc. In FIG. 4, ABA components or ABA masterbatch and polymeric materials are homogenized in a mixture (401) before being subjected to an extruder for extrusion (402). After extrusion, the extruded ABA-polymeric material blend is cooled and granulated (403) to obtain ABA-incorporated polymeric materials (405). The ABA-incorporated polymeric materials can be made in different product forms by shaping such as film, bottom, sheet, etc. (407). The ABA-polymeric material blend after extrusion can also be directly subjected to an existing common plastic processing production line (404) to obtain different product forms such as film, bottle, sheet, etc. containing the ABA-incorporated polymeric material (406).

### EXAMPLES

### Example 1. ABA-Incorporated Polymeric Low-Density Polyethylene (LDPE)

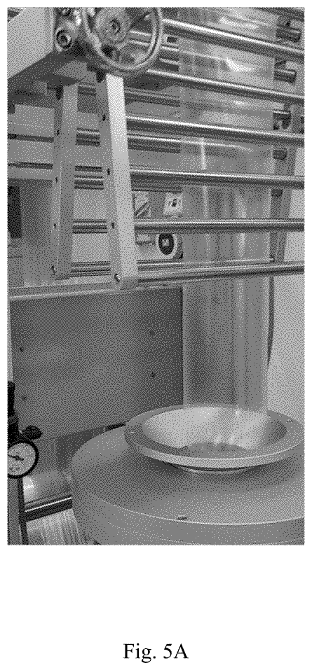

As shown in FIGS. 5A-5C, 5 wt. % ABA was mixed with low-density polyethylene (LDPE) resin and then applied for extrusion-film blowing process directly under operating temperature of 160-190° C. As shown in FIG. 5A, during the extrusion-film blowing process, the ABA-incorporated (5 wt. %) LDPE film shows good ductility with similar thickness compared to normal LDPE film. Further, ABA-incorporated LDPE film is also easy to be stored. As shown in FIG. 5B, ABA-incorporated LDPE film can be carried along as a roll like normal LDPE. In other words, the incorporation of ABA does not affect the basic mechanical properties of LDPE. Furthermore, as shown in FIG. 5C, plastic bag made of the ABA-incorporated LDPE roll has a transparent appearance. It shows that the incorporation of ABA does not change the transparency of LDPE. Therefore, the ABA incorporation does not affect or change the applicability of LDPE.

### Example 2. Production Examples of ABA Incorporated Polymeric Materials

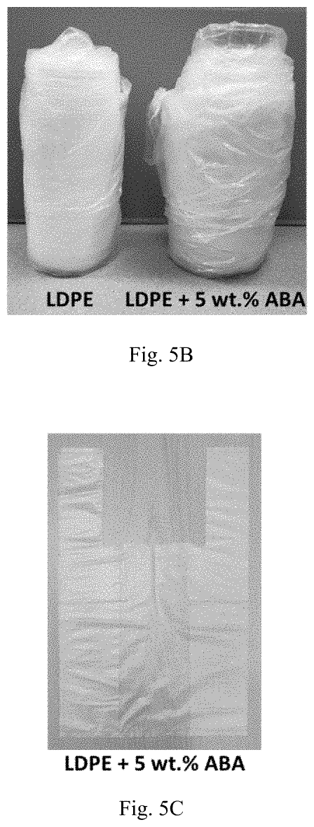

As shown in FIG. 6, different ABA incorporated polymeric materials are processed to manufacture products in variety application form. In brief, 5 wt. % ABA was mixed with POM resin and then extruded at 175-190° C., and it was then pelletized as resin of POM with ABA for further application, such as injection molding for buckle; 5 wt. % ABA was mixed with PS resin and then extruded at 185-210° C., and it was then pelletized as resin of PS with ABA for further application, such as injection molding for cutleries, and sheet production and thermoforming for cup lid; 5 wt. % ABA was mixed with PP resin and then extruded at 185-230° C., and it was then pelletized as resin of PP with ABA for further application, such as injection molding for cutleries, and sheet production and thermoforming for lunch box; 5 wt. % ABA was mixed with PET resin and then extruded at 250-270° C., and it was then pelletized as resin of PET with ABA for further application, such as blowing molding for bottles. Other product is like an EPS foamed cup with 5 wt. % ABA. The ABA-incorporated polymeric materials produced according to certain embodiments of the present invention are recyclable which is comparable to the polymeric material without ABA. It also ensures that the incorporation of ABA does not affect applicability and usability of polymeric materials.

### Example 3. Food Contact Safety Test of ABA-Incorporated Polymeric Materials

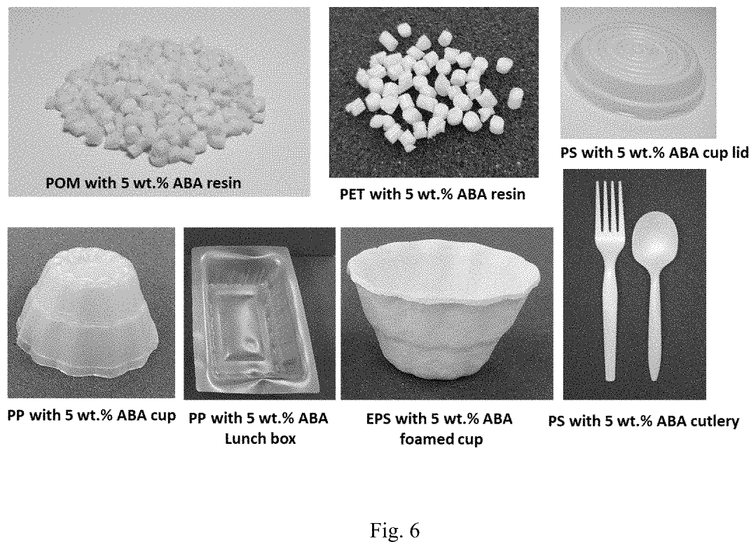

In certain embodiments, the amount of the ABA incorporated into the polymeric materials is from greater than 0% to approximately 30% by weight of the total ABA-incorporated polymeric materials, and preferably from approximately 1 to 5% by weight of the total ABA-incorporated polymeric materials, in order to achieve a minimal affection of mechanical properties compared to the polymeric materials without the ABA or when it is after recycled, and a minimal affection of properties of the original polymeric material including food contact safety when they are used in food contact safe products such as cutleries, lunch boxes, cups and cup lids. Therefore, a fork made of PS with 5 wt. % ABA was subjected to safety evaluation. As shown in FIG. 7, the PS fork with 5 wt. % ABA passed the US FDA 21 CFR 175.300 (Resinous and Polymeric Coatings)-Determination of Amount of Extractives, which means that the products made of ABA-incorporated polymeric materials are safe to be used as food contact products.

### Example 4. Mechanical Properties of ABA-Incorporated Polymeric Materials

The mechanical properties of ABA-incorporated polymeric materials are further evaluated. In brief, a LDPE film with 5 wt. % ABA showed 14 MPa tensile strength, similar to a LDPE film without the ABA having a tensile strength of 12 MPa. In other example, the POM with 5 wt. % ABA resin showed 52 MPa tensile strength while the POM resin without the ABA showed 56 MPa tensile strength. In another example, the PS with 5 wt. % ABA resin showed 1868 MPa flexural module while the PS resin without ABA showed 1635 MPa flexural module. In yet another example, the PP with 5 wt. % ABA resin showed 1940 MPa flexural module while the PP resin without ABA showed 1878 MPa flexural module. As a result, the addition of ABA does not affect the mechanical properties of polymeric materials.

### Example 5. Biodegradation Efficacy of Incorporation of ABA in Polymeric Materials

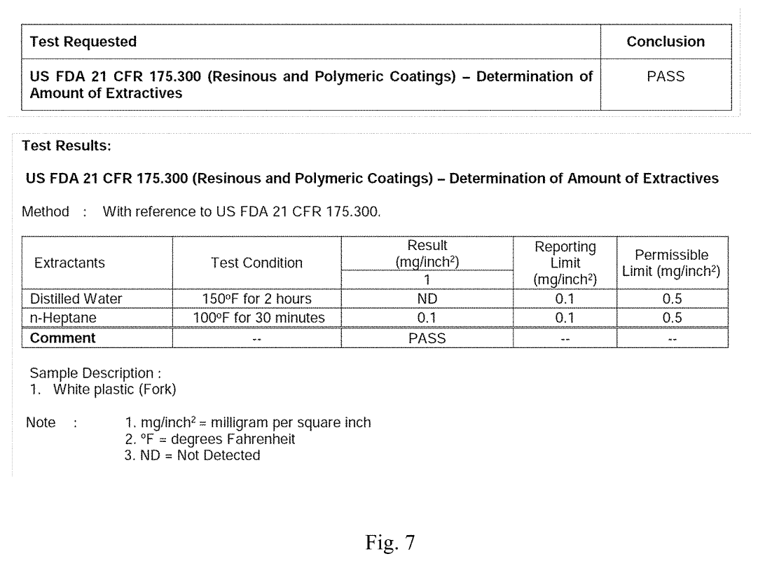

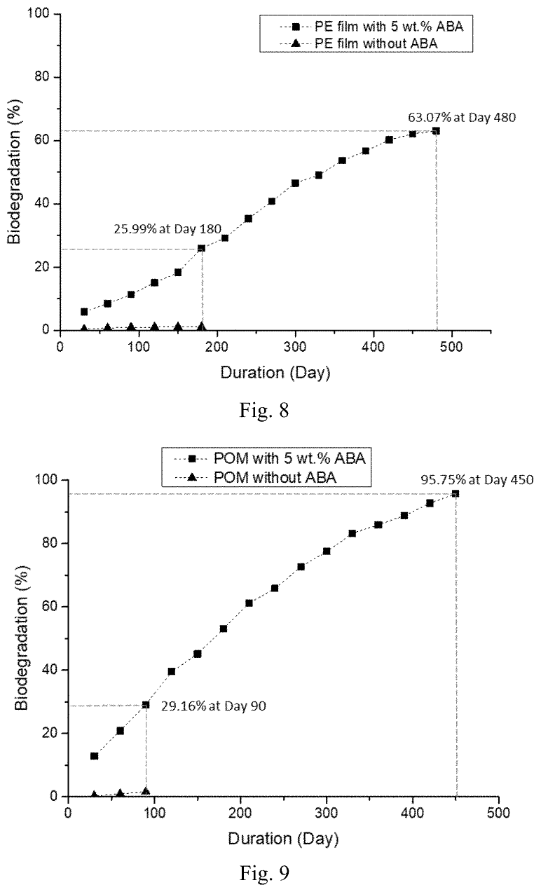

The polymeric materials with the ABA of the present invention are more biodegradable than the polymeric materials without ABA. As shown in FIG. 8, under the ASTM D5511 tests conducted by Intertek, a PE film with 5 wt. % ABA showed 25.99% biodegradation at Day 180 and 63.07% biodegradation at Day 480, while a PE film without ABA showed only 1.09% biodegradation at Day 180. As shown in FIG. 9, under the ASTM D5511 tests conducted by Intertek, POM resins with 5 wt. % ABA showed 29.16% biodegradation at Day 90 and 95.75% biodegradation at Day 450, while POM resins without ABA showed only 1.53% biodegradation at Day 90.

Other embodiments show that, under the ASTM D5511 tests conducted by Intertek, a PS cutlery with 5 wt. % ABA showed 11.67% biodegradation at Day 90 and 33.13% biodegradation at Day 270, while a PS cutlery without ABA showed only 0.08% biodegradation at Day 90; a PS cup lid with 5 wt. % ABA showed 10.50% biodegradation at Day 90, while a PS cup lid without ABA showed only 0.78% biodegradation at Day 90; a EPS foamed cup with 5 wt. % ABA showed 13.31% biodegradation at Day 90, while a EPS foamed cup without ABA showed only 0.20% biodegradation at Day 90; a PET bottle with 5 wt. % ABA showed 6.36% biodegradation at Day 45, while a PET bottle without ABA showed only 0.00% biodegradation at Day 45; a PP cup with 5 wt. % ABA showed 7.13% biodegradation at Day 45, while a PP cup without ABA showed only 0.05% biodegradation at Day 45; a PP lunch box with 5 wt. % ABA showed 7.57% biodegradation at Day 45, while a PP lunch box without ABA showed only 0.07% biodegradation at Day 45.

Without departing from the spirit and objectives of the present invention, the composition of the ABA may vary according to different applications, and the following one or more factors/criteria: biodegradability, compatibility of ABA and the polymeric materials, hydrophobicity of the polymeric materials, compatibility of the processing temperature, inference of the mechanical properties, inference of thermo-properties, inference of appearance, inference of odor, food contact safety requirements, etc.

It should be apparent to practitioner skilled in the art that the foregoing examples of the system and method are only for the purposes of illustration of working principle of the present invention. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

### INDUSTRIAL APPLICABILITY

The present ABA significantly enhances biodegradation rate of polymeric materials in anaerobic environment, and does not impact significantly on mechanical properties, recyclability and other properties of the original polymeric material including food contact safety when they are used in food contact safe products such as cutleries, lunch boxes, cups, and cup lids.

1. An anaerobic biodegradation accelerator for a host polymeric material, comprising:
a carrier matrix for gathering all other ingredients in an accelerator and assisting in dispersing them into a host polymeric material,
at least one biotic component for initiating the biodegradation of the host polymeric material,
a protective layer for protecting the biotic component and increasing shelf-life of the anaerobic biodegradation accelerator;
a biodiversity promotor for promoting and sustaining growth of the at least one biotic component,
a surfactant for promoting the interaction of the at least one biotic component and the host polymeric material,
a compatibilizer to increase the compatibility between the anaerobic biodegradation accelerator and the host polymeric material,
an antioxidant for inhibiting oxidation reaction of the anaerobic biodegradation accelerator during manufacturing, storage, and usage,
a plasticizer, and
a properties modifier.

2. The anaerobic biodegradation accelerator of claim 1, wherein the carrier matrix is in an amount of 30% to 90% of the total weight of the anaerobic biodegradation accelerator and the carrier matrix comprises biodegradable and/or non-biodegradable materials selected from one or more of polyethylene (PE), polypropylene (PP), poly (ethylene-vinyl acetate) (EVA), polystyrene (PS), polyoxymethylene (POM), polyethylene terephthalate (PET), polyethylene terephthalate glycol (PETG), polyamides (PA), polycarbonate (PC), polyurethanes (PU), thermoplastic elastomer (TPE), cellulose acetate (CA), polyvinyl chloride (PVC), acrylonitrile butadiene styrene (ABS), polyvinyl alcohol (PVA), polylactic acid (PLA), polyhydroxyalkanoates (PHAs), polybutylene succinate (PBS), polycaprolactone (PCL), polybutylene adipate terephthalate (PBAT), polyglycolic acid (PGA), and poly(lactic-co-glycolic acid) (PLGA), or any combination thereof.

3. The anaerobic biodegradation accelerator of claim 1, wherein the at least one biotic component is in an amount of greater than 0% to 20% of the total weight of the anaerobic biodegradation accelerator and the at least one biotic component is selected from bacteria, fungi, enzymes, or any combination thereof.

4. The anaerobic biodegradation accelerator of claim 3, wherein the bacteria comprises Clostridium thermocellum, Micrococcus luteus, Rhodococcus rhodochrous, Streptomyces badius, Acinetobacter spp., Alcaligenes spp., Amycolatopsis spp., Arthrobacter spp., Bacillus spp., Citrobacter spp. Corynebacterium spp., Enterobacter spp., Exiguobacterium spp., Lysinibacillus spp., Bacillus megaterium, Bacillus subtilis, Microbacterium spp., Micrococcus spp., Nocardia spp., Paenibacillus spp., Pseudomonas spp., Rhodococcus spp., Schlegelella spp., Sphingobacterium spp., and Staphylococcus spp.

5. The anaerobic biodegradation accelerator of claim 3, wherein the fungi comprise yeast, Aspergillus niger, Acremonium spp., Aspergillus spp., Aureobasidium spp., Cladosporium spp., Fusarium spp., Glioclodium spp., Mucor spp., Penicillium spp., Pestalotiopsis spp., Phanerochaete spp., Streptomyces spp. Trametes spp., and Trichoderma spp.

6. The anaerobic biodegradation accelerator of claim 3, wherein the enzymes comprise α-amylase, catalase, cellulase, cutinase, depolymerase, esterase, glucosidases, hydrolase, laccase, lipase, manganese peroxidase, urease, protease such as papain, bromelain.

7. The anaerobic biodegradation accelerator of claim 1, wherein the protective layer is in an amount of greater than 0% to 30% of the total weight of the anaerobic biodegradation accelerator and the protective layer comprises one or more protective layer materials of gum arabic, sodium alginate, gelatin, chitosan, cellulose, polyvinyl alcohol, poly(lactic-co-glycolic acid), polyethylene glycol, or any combination thereof, and further incorporates the surfactant.

8. The anaerobic biodegradation accelerator of claim 1, wherein the biodiversity promotor is in an amount of greater than 0% to 20% of the total weight of the anaerobic biodegradation accelerator and the biodiversity promotor comprises saccharide compounds, nitrogen-containing compounds, phosphorous compounds, or any derivatives thereof, and micronutrients, and wherein the saccharide compounds comprise cyclodextrins, cellulose, starch, sucrose, and glucose; the nitrogen-containing compounds comprise proteins, meat extracts, autolysates, nitrates, and urea; the phosphorous compounds comprise phosphorus pentoxide, hydrogen phosphates, dihydrogen phosphate, and organic phosphate; the derivatives comprise pectin, xylan, carboxylic acids, amino acids; and the micronutrients comprises vitamins, minerals, potassium, calcium, magnesium, iron, manganese, zinc, boron, copper, and molybdenum; or any combination thereof

9. The anaerobic biodegradation accelerator of claim 1, wherein the surfactant is in an amount of greater than 0% to 10% of the total weight of the anaerobic biodegradation accelerator and the surfactant is one or more of non-ionic and ionic surfactants, and wherein the non-ionic surfactants comprise polysorbates, sorbitan esters, and alkylphenol ethoxylates; the ionic surfactants comprises cationic surfactants, anionic surfactants, zwitterionic surfactants, and biosurfactants, and wherein the anionic surfactants comprise anionic functional group-containing compounds comprises sulfate, sulfonate, phosphate, carboxylate derivatives, prominent alkyl sulfates include ammonium lauryl sulfate, sodium lauryl sulfate and the related alkyl-ether sulfates sodium laureth sulfate and sodium myreth sulfate, dioctyl sodium sulfosuccinate, perfluorooctanesulfonate, perfluorobutanesulfonate, alkyl-aryl ether phosphates, alkyl ether phosphates sodium dodecylbenzenesulfonate, sodium dodecyl sulfate, sodium stearate, calcium stearate; the cationic surfactants comprise octenidine dihydrochloride, cetrimonium bromide, cetylpyridinium chloride, benzalkonium chloride, benzethonium chloride, dimethyldioctadecylammonium chloride, and dioctadecyldimethylammonium; the zwitterionic surfactants comprise lauryldimethylamine oxide and myristamine oxide; the biosurfactants comprise glycolipids, phospholipids, lipopeptides, neutral lipids, fatty acids, and lipopolysaccharides; or any combination thereof.

10. The anaerobic biodegradation accelerator of claim 1, wherein the compatibilizer is in an amount of greater than 0% to 10% of the total weight of the anaerobic biodegradation accelerator and the compatibilizer comprises chain extenders and coupling agents, and wherein the chain extenders comprise modified styrene acrylic polymers, lactic acid, ethylene glycol, and 1,4-butanediol; the coupling agents comprise maleic anhydride, Tung oil anhydride, epoxidized soybean oil, methylene-diphenyldiisocyanate, acrylic acid, and citric acid; or any combination thereof.

11. The anaerobic biodegradation accelerator of claim 1, wherein the plasticizer is in an amount of greater than 0% to 10% of the total weight of the anaerobic biodegradation accelerator and the plasticizer comprises water, urea, glycerol, ethylene glycol, polyethylene glycol, Tung oil anhydride, epoxidized soybean oil, triethyl citrate, and acetyl triethyl citrate, or any combination thereof

12. The anaerobic biodegradation accelerator of claim 1, wherein the properties modifier is in an amount of greater than 0% to 10% of the total weight of the anaerobic biodegradation accelerator and the properties modifier comprises calcium carbonate, titanium dioxide, talcum powder, organomontmorillonite, bentonite, nanofillers, natural fiber, color masterbatch, and scent masterbatch, or any combination thereof.

13. The anaerobic biodegradation accelerator of claim 1, wherein the antioxidant is in an amount of greater than 0% to 10% of the total weight of the anaerobic biodegradation accelerator and the antioxidant comprises ascorbic acid, tocopherols, glutathione, tetrakis [methylene(3,5-di-t-butyl-4-hydroxyhydrocinnamate)]methane, tris(2,4-di-tert-butylphenyl) phosphite, lipoic acid, and uric acid, or any combination thereof.

14. The anaerobic biodegradation accelerator of claim 1, wherein the host polymeric material comprises polyethylene (PE), polypropylene (PP), poly (ethylene-vinyl acetate) (EVA), polystyrene (PS), polyoxymethylene (POM), polyethylene terephthalate (PET), polyethylene terephthalate glycol (PETG), polyamides (PA), polycarbonate (PC), polyurethanes (PU), thermoplastic elastomer (TPE), cellulose acetate (CA), polyvinyl chloride (PVC), acrylonitrile butadiene styrene (ABS), polyvinyl alcohol (PVA), polylactic acid (PLA), polyhydroxyalkanoates (PHAs), polybutylene succinate (PBS), polycaprolactone (PCL), polybutylene adipate terephthalate (PBAT), polyglycolic acid (PGA), and poly(lactic-co-glycolic acid) (PLGA), or any combination thereof, and wherein the host polymeric material is conventional plastic or any processed plastic by processes including extrusion, resin making, foaming, sheet production, thermoforming, injection molding, film blowing, blow molding, fiber/fabric and filament making.

15. A method for producing the anaerobic biodegradation accelerator of claim 1 as a masterbatch, comprising:
introducing the protective layer material to the at least one biotic component to form a protective layer surrounding the at least one biotic component;
homogenizing the at least one biotic component with protective layer and the remaining components and/or materials of the anaerobic biodegradation accelerator at a first elevated temperature to obtain a mixture;
extruding the mixture under a second elevated temperature until the masterbatch is obtained.

16. The method of claim 15, wherein the first elevated temperature ranges from room temperature to about 80° C. and the second elevated temperature ranges from 50° C. to about 250° C.

17. The method of claim 15, wherein the homogenization is carried out at a mixing speed of 40 to 1,000 rpm.

18. A method for producing an anaerobic biodegradation accelerator-incorporated polymeric material incorporated with the anaerobic biodegradation accelerator of claim 1 in a masterbatch form, comprising:
homogenizing the masterbatch of the anaerobic biodegradation accelerator with a host polymeric material to form a blend;
extruding the blend at a third elevated temperature to obtain an anaerobic biodegradation accelerator-incorporated polymeric material.

19. The method of claim 18, wherein the anaerobic biodegradation accelerator is in an amount of greater than 0% to 30% by weight of the total anaerobic biodegradation accelerator-incorporated polymeric material.

20. The method of claim 18, wherein the anaerobic biodegradation accelerator is in an amount of about 1% to 5% by weight of the total anaerobic biodegradation accelerator-incorporated polymeric material in order to achieve a minimal affection of mechanical properties, and other properties of the original polymeric material including food contact safety when they are used in food contact safe products.

21. The method of claim 18, wherein the anaerobic biodegradation accelerator-incorporated polymeric material has a recyclability comparable to that of the host polymeric material.

22. The method of claim 18, wherein the anaerobic biodegradation accelerator-incorporated polymeric material has a superior biodegradability than that of the host polymeric material.

23. The method of claim 18, wherein the host polymeric material comprises polyethylene (PE), polyoxymethylene (POM), polystyrene (PS), expanded polystyrene (EPS), polypropylene (PP) and polyethylene terephthalate (PET) incorporated with the anaerobic biodegradation accelerator.

24. The method of claim 18, wherein the third elevated temperature ranges from 50° C. to about 300° C.

25. The method of claim 18, wherein the homogenization is carried out at a mixing speed of 40 to 1,000 rpm.
